(19) United States
(12) Patent Application Publication
(10) Pub. No.: US 2019/0183420 A1
(43) Pub. Date: Jun. 20, 2019

(54) SKIN OBSERVATION SYSTEM

(71) Applicant: OMM IMPORTS INC., Doral, FL (US)

(72) Inventor: MOTI SHENFARBER, MIAMI BEACH, FL (US)

(21) Appl. No.: 15/844,041

(22) Filed: Dec. 15, 2017

(51) Int. Cl. A61B 5/00; A42B 3/04; A61B 90/50; A42B 3/28

(52) U.S. Cl. CPC A61B 5/6803; A61B 5/0079; A61B 5/441; A42B 3/042; A61B 5/7475; A61B 90/50; A42B 3/286; A42B 3/044

(57) ABSTRACT

A skin observation system includes a helmet to be placed over the head of a user, a mirror disposed within the helmet in a direct line of sight of the user wearing the helmet, at least one lighting device disposed within the helmet for illuminating the face of the user, a shield disposed within the helmet for shielding the eyes of the user from light emitted by the at least one lighting device, and a filter disposed within the helmet for filtering components of the light emitted by the at least one lighting device. The lighting device is preferably a plurality of laser diodes and the filter is preferably a UV filter. The mirror preferably has a concave surface facing the user.

### BACKGROUND OF THE INVENTION

### Field of the Invention

The invention relates to a skin observation system which permits a user to observe skin conditions, such as acne, on the user's own face.

### Description of the Related Art

U.S. Patent Publication No. 20160058157 A1 discloses a headband having a concave mirror for viewing one's own ear. U.S. Patent Publication No. 20080260243 A1 teaches a helmet having a digital camera for recording the color of the hair. U.S. Patent Publication No. 20070002479 A1 shows a device having a flashlight, lenses, filters and a mirror for viewing the skin. Goggles are worn by the user. U.S. Pat. No. 7,532,746 B2 discloses a headset for illuminating the skin with IR and locating blood vessels. U.S. Pat. No. 7,004,599 B2 teaches a hand-held mirror having a polarizer and a light source in a frame covered by a polarizing filter. The two polarizers are out of phase. U.S. Pat. No. 5,760,407 shows a device having a UV light source and a filter for identifying acne in fluorescing skin follicles. U.S. Pat. No. 5,047,006 discloses a sphere to be worn on the head and providing illumination and sound within the sphere. U.S. Pat. No. 4,911,544 teaches a shade or casing covering the head of a user and having a dark interior, a concave mirror and light sources for illuminating the skin of the user. A cosmetologist can look though a hole in the shade and a camera 46 can take pictures of the user.

None of the prior art references provide measures for adequately shielding or filtering light so that it is not harmful to the eyes of the user.

### SUMMARY OF THE INVENTION

It is accordingly an object of the invention to provide a skin observation system, which overcomes the hereinafore-mentioned disadvantages of the heretofore-known devices of this general type and which provides the user with the ability to observe and understand his or her skin condition with the user's own eyes, without the intermediary of cameras and virtual reality elements and which adequately protects the eyes of the user.

With the foregoing and other objects in view there is provided, in accordance with the invention, a skin observation system, comprising a helmet to be placed over the head of a user, a mirror disposed within the helmet in a direct line of sight of the user wearing the helmet, at least one lighting device disposed within the helmet for illuminating the face of the user, a shield disposed within the helmet for shielding the eyes of the user from light emitted by the at least one lighting device, and a filter, such as an ultraviolet filter, disposed within the helmet for filtering components of the light emitted by the at least one lighting device.

Acne and other skin problems are exacerbated by microorganisms that can be seen under fluorescent or ultra violet light, using the filter and the mirror of the invention to reflect an image. The device is made portable by virtue of being built within a helmet that the user places on his or her head. The observation of the user is made possible by a combination of filtered laser light that illuminates a specific facial area and the result of this action is seen by the user in the self-image that appears on the mirror in the helmet.

In accordance with another feature of the invention, the mirror has a concave surface directed toward the face of the user. The mirror is adjustable in position toward and away from the face of the user so that different users can be accommodated. The concavity is provided to focus the eyes of the user, to make the image in the mirror appear larger and to make the vision field narrower to prevent the user from seeing reflections of and flashes of light.

In accordance with a further feature of the invention, the lighting device includes a plurality of laser diodes disposed along two rows, one of the rows is disposed below and another of the rows is disposed above the mirror, so they will illuminate the entire relevant part of the face without shadows. The laser diodes provide a narrow spectral strip, they do not focus but they do use a light spreading filter. The laser diodes raise the signal-to-noise ratio and allow the user's own eyes to see the true condition of the skin through simple filters.

In accordance with an added feature of the invention, at least one camera is mounted within the helmet for recording images on the mirror. This permits the images shown on the mirror to be seen by an operator or recorded, such as for comparison with other images.

In accordance with an additional feature of the invention, a keypad or buttons are mounted on an outer surface of the helmet for controlling the at least one lighting device and/or the at least one camera. This permits an operator to control the system.

In accordance with yet another feature of the invention, a transmitter is disposed on the helmet, and a remote control device communicates with the transmitter for controlling the at least one lighting device and/or the at least one camera. This also permits an operator to control the system. The remote control device may be a smart phone running an application for receiving, displaying and storing the images on the mirror. The remote control and the transmitter may communicate by NFC (Near Field Communication), Bluetooth or WiFi.

In accordance with yet a further feature of the invention, a stand is provided on which the helmet is mounted. The stand includes at least one joint for adjusting a position of the helmet for the user and/or at least one telescoping arm connected to the at least one joint for additional adjustment of the position of the helmet for the user.

In accordance with yet an additional feature of the invention, the ultraviolet light filter has a concave surface directed toward the face of the user, one end attached to an inner surface of the helmet, and a free end extending below the line of sight of the user wearing the helmet. The filter protects the eyes from light in the ultraviolet region of the electromagnetic spectrum.

In accordance with again another feature of the invention, a ventilator is disposed on the helmet for cooling an inner area of the helmet during operation.

In accordance with a concomitant feature of the invention, a preferably black lining extends entirely over an inner surface of the helmet for absorbing light and preventing reflections of light.

Other features which are considered as characteristic for the invention are set forth in the appended claims.

Although the invention is illustrated and described herein as embodied in a skin observation system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING:

FIG. 1 is a diagrammatic, side-elevational view of the skin observation system according to the invention being employed by a user to observe the user's skin condition;

FIG. 2 is a top-plan view of the skin observation system being employed by the user;

FIG. 3 is a front-elevational view of the skin observation system being employed by the user; and

FIGS. 4A, 4B and 4C are respective top-plan, side-elevational and front-elevational views of a stand on which the helmet of the invention is disposed.

### DESCRIPTION OF THE PREFERRED EMBODIMENTS

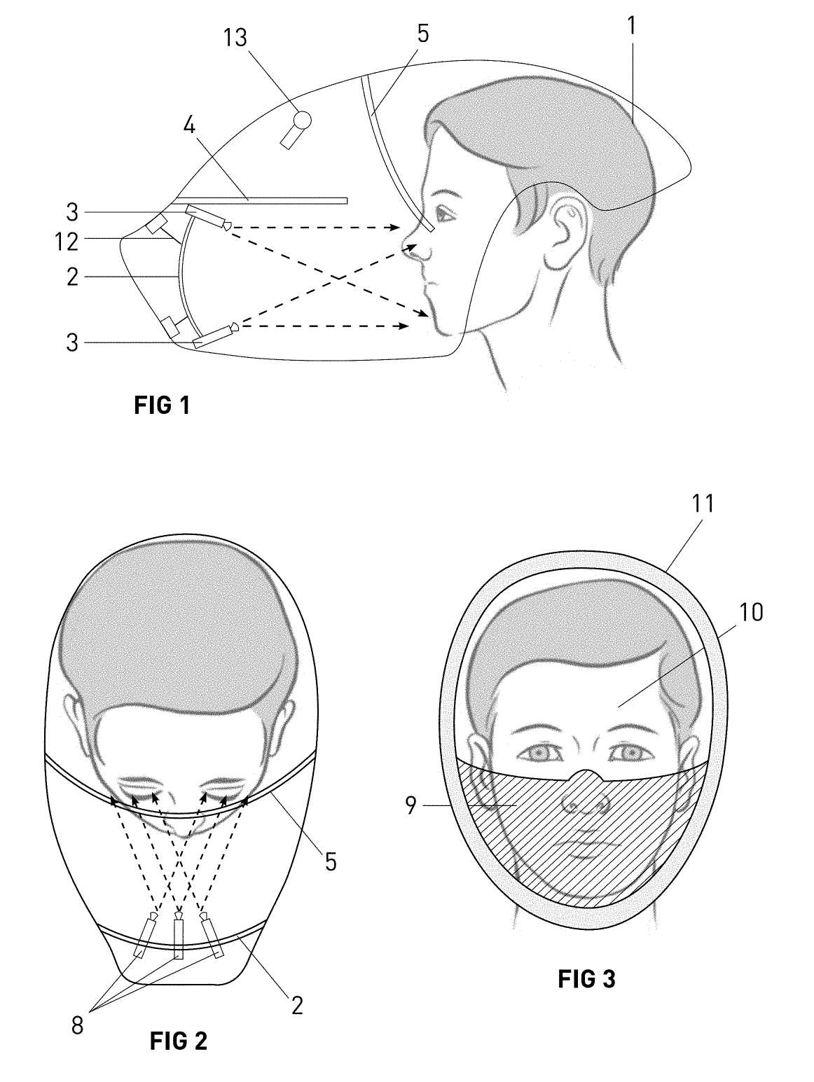

Referring now to the figures of the drawings in detail and first, particularly, to FIG. 1 thereof, there is seen a helmet 1 which is placed on the head of a user. The helmet 1 is illustrated in the figures as being transparent so as to show the elements contained therein but in practice the helmet is opaque so that light cannot pass through the helmet. The purpose of the helmet is to act as a frame for an electro-optical system disposed inside the helmet and to ensure that the environment inside the helmet is completely dark.

A concave mirror 2 is placed inside the helmet 1 in a direct line of sight of the user wearing the helmet. The mirror is concave for three reasons: to focus the eyes of the user over a very short distance which is dictated by the size of the helmet, to make the images in the mirror appear larger than they actually are (similar to a makeup mirror), and to make the vision field narrower, so that the user will not see reflections and flashes of light, thus allowing images seen by the user to have greater contrast.

Laser diodes 3 emit laser beams which are directed toward the user's face. The laser diodes provide a narrow spectral strip, they do not use a focusing lens but they do use a light spreading filter. Therefore, the laser diodes are without the ability to focus. The laser diodes raise the signal-to-noise ratio and allow the user's own eyes to see the true condition of the skin through simple filters. The laser light emitted by the laser diodes cannot reach the eyes of the user due to the fixed position of the laser diodes in the device. The laser diodes are disposed along two lines, an upper line and a lower line, so they will illuminate the entire relevant part of the face without shadows such as from the nose, for example. Only the lower line 8 of the laser diodes 3 is shown in FIG. 2.

An inner eye shield 4 prevents flashes of light from reaching the eyes of the user. A filter 5 blocks UV radiation from reaching the eyes.

FIG. 2 shows the mirror 2 and the filter 5 from above. FIG. 2 also shows the laser diodes 3 disposed along a lower row 8. It can be seen that the filter 5 also filters all of the light emitted by the lower row 8 of laser diodes 3 before reaching the eyes.

FIG. 3 indicates that an area 9 is illuminated by the laser diodes 3, whereas an area 10 is not illuminated by the laser diodes 3 because it is screened by the filter 5, although glasses may also be used for filtering. FIG. 3 also shows that the helmet has a lining 11 extending entirely over the inner surface of the helmet. The lining is made from a material having a black color that absorbs the light and prevents reflections.

During the operation of the system, the user may mix the light emitted from the laser diodes 3 between blue light and white light, in order to give the user the option of seeing his or her face in the mirror in “normal” white light and then in a blue light for observation.

The laser diodes 3 and the ventilator may be provided with energy in various ways, by using a rechargeable battery, a non-rechargeable battery or USB cable from a computer, a wall outlet or another source.

The mirror 2 is mounted on adjustment screws 12 so that it may be moved closer to or farther from the face of the user.

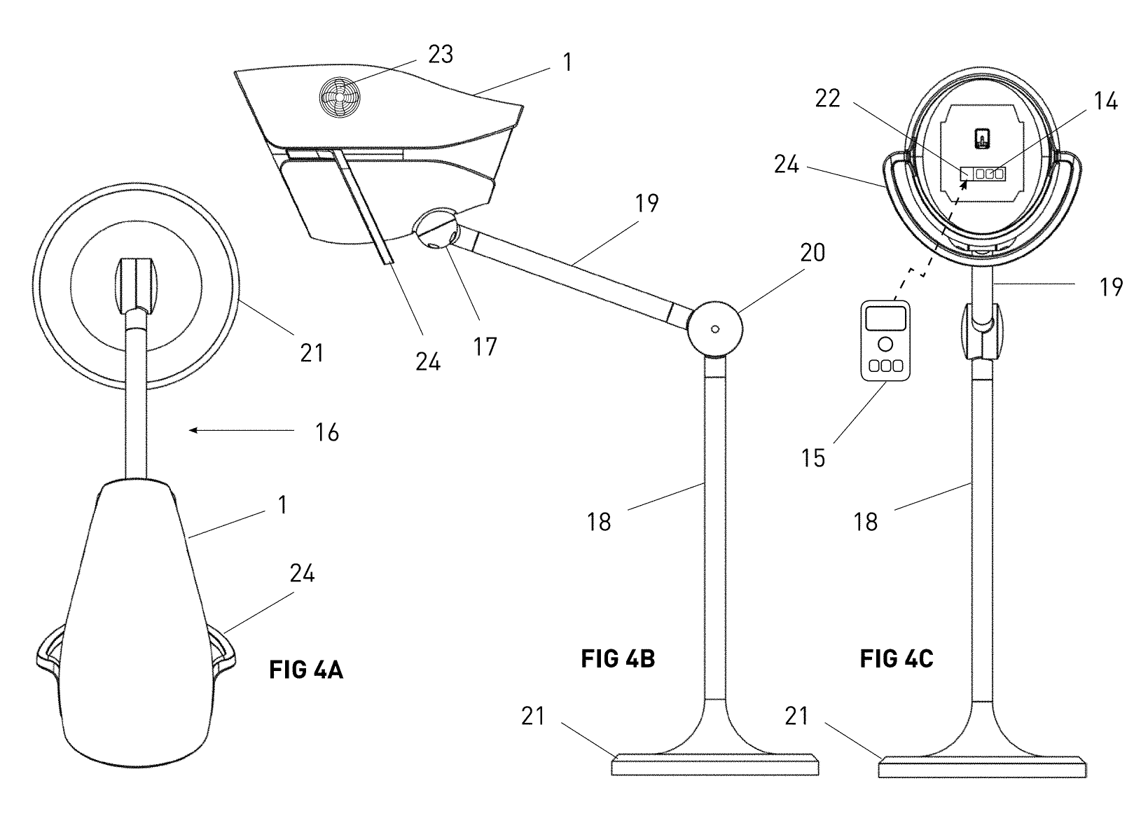

The helmet 1 can be provided with a supporting stand 16 as shown in FIGS. 4A-4C, so that the stand and not the user carries the weight of the helmet. FIG. 4B shows that the helmet has been retracted upwardly around a joint 17, so that the user may sit under the helmet and bring it down over the user's head, as is done with a drier in a beauty salon. A handle 24 facilitates this movement. The stand 16 has a base 21 and two arms 18, 19 which pivot around a joint 20 for adjustment of the position of the helmet. One or both of the arms 18, 19 may be telescopic for additional adjustment. FIG. 4B also shows that a ventilator 23 may be provided in the helmet 1 to cool the inner area thereof while operating the system.

At least one camera 13 may also be provided in the helmet in order to take photographs of the image in the mirror. It is recommended that an operator of the system should be near the user at all times. The operator may operate the system by using a keypad 14 having buttons or buttons without a keypad on the helmet or on a remote control device, which may be a dedicated remote control or a smart phone 15 running an app as seen in FIG. 4C. The keypad 14 may be located anywhere on the helmet or the stand which is accessible from outside the helmet. The keypad 14 or buttons or app on the smart phone 15 can operate the lasers diodes 3 and the camera 13. The remote control or smart phone may receive, display and store the images shown on the mirror, such as for later comparison to other images of the user or to images of stored skin conditions. The photographs may be transmitted through a cable or wirelessly, such as by NFC, Bluetooth or WiFi from a transmitter 22. The pictures may also be saved on storage device, such as a thumb drive, and displayed on a computer screen or a television.

Safe operation of the system is ensured because the helmet will automatically be in the correct position when the user's head touches two points in the helmet. The user's forehead will touch the upper part of the helmet and the user's chin will touch the lower part of the helmet, ensuring correct positioning before the laser diodes are activated. In this way, it is not possible for the laser diodes to be activated before the head of the user is in the correct location and protected by the shield and filters.

1. A skin observation system, comprising:
a helmet to be placed over the head of a user;
a mirror disposed within said helmet in a direct line of sight of the user wearing said helmet;
at least one lighting device disposed within said helmet for illuminating the face of the user;
a shield disposed within said helmet for shielding the eyes of the user from light emitted by said at least one lighting device; and
a filter disposed within said helmet for filtering components of the light emitted by said at least one lighting device.

2. The skin observation system according to claim 1, wherein said mirror has a concave surface facing the face of the user.

3. The skin observation system according to claim 2, wherein said mirror is adjustable in position toward and away from the face of the user.

4. The skin observation system according to claim 1, wherein said at least one lighting device is at least one laser diode.

5. The skin observation system according to claim 4, wherein said at least one laser diode includes a plurality of laser diodes disposed along at least one row.

6. The skin observation system according to claim 5, wherein said plurality of laser diodes are disposed along two rows, one of said rows is disposed below and another of said rows is disposed above said mirror.

7. The skin observation system according to claim 1, which further comprises at least one camera mounted within said helmet for recording images on said mirror.

8. The skin observation system according to claim 1, which further comprises a keypad or buttons mounted on an outer surface of said helmet for controlling said at least one lighting device.

9. The skin observation system according to claim 7, which further comprises a keypad or buttons mounted on an outer surface of said helmet for controlling said at least one lighting device and said at least one camera.

10. The skin observation system according to claim 1, which further comprises a transmitter disposed on said helmet, and a remote control device communicating with said transmitter for controlling said at least one lighting device.

11. The skin observation system according to claim 7, which further comprises a transmitter disposed on said helmet, and a remote control device communicating with said transmitter for controlling said at least one lighting device and said at least one camera.

12. The skin observation system according to claim 11, wherein said remote control device is a smart phone running an application for receiving, displaying and storing the images on said mirror.

13. The skin observation system according to claim 1, which further comprises a stand on which said helmet is mounted, said stand including at least one joint for adjusting a position of said helmet for the user.

14. The skin observation system according to claim 13, wherein said stand includes at least one telescoping arm connected to said at least one joint for additional adjustment of the position of said helmet for the user.

15. The skin observation system according to claim 13, wherein said filter is an ultraviolet light filter.

16. The skin observation system according to claim 13, wherein said ultraviolet light filter has a concave surface facing the face of the user, one end attached to an inner surface of said helmet, and a free end extending below the line of sight of the user wearing said helmet.

17. The skin observation system according to claim 1, which further comprises a lining extending entirely over an inner surface of the helmet for absorbing light and preventing reflections of light.

18. The skin observation system according to claim 1, which further comprises a ventilator disposed on said helmet for cooling an inner area of said helmet during operation.
